(19) United States
(12) Patent Application Publication
(10) Pub. No.: US 2007/0081622 A1
(43) Pub. Date: Apr. 12, 2007

(54) Method for scattered radiation correction of a CT system

(76) Inventors: Herbert Bruder, Hochstadt (DE); Martin Petersilka, Adelsdorf (DE)

Correspondence Address: HARNESS, DICKEY & PIERCE, P.L.C., P.O.BOX 8910, RESTON, VA, 20195, US

(21) Appl. No.: 11/543,930

(22) Filed: Oct. 6, 2006

(30) Foreign Application Priority Data
Oct. 10, 2005 (DE) 10 2005 048 388.7

(51) Int. Cl. H05G 1/60; A61B 6/00; G01N 23/00; G21K 1/12

(52) U.S. Cl. 378/7

(57) ABSTRACT

A method is disclosed for scattered radiation correction of a CT system including two simultaneously operated focus/detector systems, arranged angularly offset from one another on a rotatable gantry. In an embodiment of the method, in order to scan an object, the two focus/detector systems arranged angularly offset from one another scan the object by virtue of the fact that they rotate about a system axis of the CT system. A multiplicity of absorption values of individual rays are then determined from the measured attenuations of the radiation of the foci and the measured values are subjected to scattered radiation correction. The positive differences for the direct rays are determined in channelwise fashion from the intensity values of the direct rays and the intensity values of the “complementary” rays removed by 180°, and this positive difference is subtracted as scattered radiation correction from the intensity value of the direct ray to determine the attenuation values and to thereafter reconstruct CT tomograms or CT volume data.

### PRIORITY STATEMENT

The present application hereby claims priority under 35 U.S.C. §119 on German patent application number DE 10 2005 048 388.7 filed Oct. 10, 2005, the entire contents of which is hereby incorporated herein by reference.

### FIELD

The invention generally relates to a method for scattered radiation correction of a computed tomography (CT) system. For example, it may relate to one having two simultaneously operated focus/detector systems, arranged angularly offset from one another on a rotatable gantry. Further, it may relate to one in which, in order to scan an object, the two focus/detector systems arranged angularly offset scan the object by virtue of the fact that they rotate about a system axis of the CT system, and a multiplicity of absorption values of individual rays are determined from the measured attenuations of the radiation of the foci, and the measured values are subjected to scattered radiation correction in order subsequently to reconstruct CT pictures or volume data of the object with the aid of the determined absorption data.

### BACKGROUND

A method is disclosed, for example, in patent specification DE 102 32 429 B3. In the case of this patent specification, two focus/detector systems arranged angularly offset from one another are operated in an alternating fashion at least temporarily, such that the scattered radiation actually occurring that originates from the focus/detector system being operated can be measured directly in the focus/detector system respectively not switched on. In order to carry out this method, it is necessary to operate the X-ray sources in an alternating fashion at least temporarily, as a result of which at these times image information from the CT scan is lacking at least in the detector of the X-ray tube that is not being operated, and so gaps are produced in the data acquisition. This is disadvantageous, particularly in the case of CT cardio pictures, which require a high time resolution, and this method leads in practice to deficient recording results.

### SUMMARY

At least one embodiment of the invention is directed to a method for scattered radiation correction of a CT system having two focus/detector systems arranged angularly offset from one another, which method renders it possible to dispense with the direct measurement of the scattered radiation, and enables the scattered radiation fraction to be determined in continuous operation of the two focus/detector systems.

A fundamental distinction is made between forward scattering and transverse scattering in the case of scattered radiation. However, the forward scattering cancels itself out with the primary radiation, has no effect on another focus/detector system arranged in a rotationally offset fashion, and therefore is not taken into account in this application. In the sense of the application, the radiation designated as scattered radiation in the following text is always the transverse scattering of a radiation that leads to errors in the measurement of the attenuation of the direct radiation in the case of a focus/detector system arranged in a rotationally offset fashion, since it simulates an apparent reduction in the actual attenuation even if the focus/detector system arranged in a rotationally offset fashion is operating and generating scattered radiation that is measured in the detector arranged in a rotationally offset fashion.

The inventors have realized, in at least one embodiment, that during scanning of an object with the aid of two focus/detector systems arranged angularly offset from one another, a typical distribution of the scattered radiation is produced that largely renders it possible to determine the scattered radiation fraction from the measured data of rays arranged in an oppositely directed fashion in space, or from oppositely situated projections. In accordance with the realization of the inventors in at least one embodiment, what is decisive here is that the scattered radiation is not produced uniformly in the scanned object, but substantially at the surface of the object that faces the focus forming the scattered radiation. Consequently, the scattered radiation generates a strongly asymmetric profile in a projection, and this also helps explain the inhomogeneities and artifacts existing in the reconstructed CT data without scattered radiation correction.

Thus, it may be stated on the basis of this realization that when considering rays through an object that are situated identically in space it is possible to regard as the scattered radiation fraction at least the intensity fraction that is greater than the radiation intensity in the opposite direction. If this realization is extended to complete data oriented identically in space and sorted in parallel, but projections offset by 180° or π, it is correspondingly possible also to conclude from the difference between the projections that the respectively positive excess of intensity of oppositely directed projections is respectively to be ascribed to the scattered radiation of a focus/detector combination that is arranged angularly offset from the currently considered focus/detector combination.

On the basis of this fundamental idea, the inventors, in at least one embodiment, propose both a method for scattered radiation correction by considering individual oppositely directed rays of identical focus/detector systems and a different method for scattered radiation correction by considering oppositely directed parallel projections, that is to say ones that are offset by π.

In accordance with the first fundamental idea of at least one embodiment of the invention, the method known per se for scattered radiation correction of a CT system having two simultaneously operated focus/detector systems, arranged angularly offset from one another on a rotatable gantry, in which in order to scan an object the focus/detector systems arranged angularly offset from one another scan the object by virtue of the fact that they rotate about a system axis of the CT system, and a multiplicity of absorption values of individual rays are determined from the measured attenuations of the radiation of the foci, and the measured values are subjected to scattered radiation correction in order subsequently to reconstruct CT pictures or CT volume data of the object with the aid of the determined absorption data, is improved to the effect that for each direct ray of a focus/detector system, an oppositely directed complementary ray of the same focus/detector system offset by 180° is sought and, if it cannot be taken directly from the detector data, it is determined by interpolation of absorption data of rays of this focus/detector system that are situated and oriented in a spatially similar fashion, the intensity value of the complementary ray is subtracted from the attenuated intensity values of each direct ray, and if the intensity value of the direct ray is greater than the intensity value of the complementary ray this difference in the intensity values is interpreted as scattered radiation fraction and subtracted from the intensity value of the direct ray, and the corrected absorption value of the direct ray is determined therefrom, in order to reconstruct CT pictures or CT volume data from the corrected absorption values.

In accordance with a further idea of at least one embodiment of the invention, the inventors propose the improvement of a known method for scattered radiation correction of a CT system having two simultaneously operated focus/detector systems, arranged angularly offset from one another on a rotatable gantry, in which in the known method in order to scan an object the focus/detector systems arranged angularly offset from one another scan the object by virtue of the fact that they rotate about a system axis of the CT system, and there are provided from the measured attenuations of the radiation of the foci a multiplicity of parallel projections from absorption values that are calculated from the intensity values, attenuated by the object and unattenuated, and the measured values are subjected to scattered radiation correction, in order to reconstruct CT pictures of the object with the aid of the parallel projections. The improvement of this method resides in the fact that for each direct parallel projection of a focus/detector system that originates exclusively from absorption data, measured in the same direction, of a focus/detector system, an oppositely directed, complementary parallel projection of the same focus/detector system is sought and, if it cannot be taken directly from the detector data, is interpolated by interpolation of absorption data of rays of the same focus/detector system that are situated and oriented in a spatially similar fashion, subsequently the channel-wise existing differences of positive sign are interpreted as the scattered radiation fraction and are subtracted from the direct parallel projection in channel-wise fashion for the purpose of scattered radiation correction in order to reconstruct CT pictures or CT volume data from the corrected projection data.

The outcome of these two inventive variants, outlined above, of the same fundamental idea is that the scattered radiation fraction is now calculated without any loss of dose exclusively from the analytical data of a scan of an object, preferably a patient, and is subtracted from the determined intensity value of a ray, the result thereby being to achieve a substantial improvement in the CT pictures or CT volume data reconstructed from these corrected measured data.

It is to be stressed, in particular, that the described method must be carried out with the aid not of the absorption data −ln(I/I₀) but of the measured intensities I.

If this method is applied for all measured data from the focus/detector systems used, it is subsequently possible to carry out the reconstruction exclusively with the aid of absorption data of identical focus/detector systems, or it is possible to mix the absorption data of the two focus/detector systems for the reconstruction. This can be advantageous, for example, when an enhanced time resolution is desired as is the case, for example, with cardio CT pictures.

It may also be pointed out, furthermore, that a calibration can and should be carried out in the way known per se before the scattered radiation correction is carried out for each focus/detector system, for example this calibration is an air calibration, a normalization to a dose monitor value, a radiation hardening correction, a channel correction and a water scaling, as they are generally known.

In order to avoid problems owing to differences between the measurements of the two focus/detector systems, it can be advantageous when mutual fitting of the focus/detector systems is additionally carried out by mutual normalization before the measurement.

It can also be advantageous, furthermore, when the scattered radiation fractions are extrapolated in the channel region of the projections in which the signals of the scattered radiation of the direct and complementary rays cancel one another, that is to say in the region of the centrally positioned channels of the projections. For example, use may be made for the extrapolation of edge values in relation to the central channels, and knowledge of test measurements relating to the profile of the scattered radiation can be employed.

### BRIEF DESCRIPTION OF THE DRAWINGS

The invention is described in more detail below using the example embodiments and with the aid of the figures, only the features required for understanding the invention being illustrated. The following reference numerals are used here: 1: CT system; 2: first focus; 3: first detector system; 4: second focus; 5: second detector system; 6: gantry housing; 7: patient; 8: displaceable patient couch; 9: system axis; 10: control and computation unit; 11: ray fan of the X-ray tube 2; 12: ray fan of the X-ray tube 4; 13: intensity profile of the scattered radiation of a direct projection p; 14: intensity profile of the scattered radiation of a complementary projection p′; 15: channelwise difference between the two projections p and p′; Prg₁-Prgₙ: computer programs for performing the inventive method; I: intensity; I₀: initial intensity; S: direct ray; S′: complementary ray; F_{A}: focus of the focus/detector system FDSA; F_{B}: focus of the focus/detector system FDSB; D_{A}: detector of the focus/detector system FDSA; D_{B}: detector of the focus/detector system FDSB; Δ: scattered radiation fraction of the complementary ray S′; β_{A}: fan angle of the focus/detector system FDSA; β_{B}: fan angle of the focus/detector system FDSB.

In detail:

FIG. 1: shows a 3D schematic of a CT system having two focus/detector systems arranged in an angularly offset fashion;

FIG. 2: shows a schematic of a cross section through a CT system in accordance with FIG. 1;

FIG. 3: shows a simplified illustration of a direct ray through a patient with a simultaneous scattered radiation fraction from the angularly offset focus;

FIG. 4: shows an illustration from FIG. 3, but angularly offset by 180°; and

FIG. 5: shows the intensity profile of the scattered radiation in a direct parallel projection, and one complementary thereto, including the profile of the difference formation.

### DETAILED DESCRIPTION OF THE EXAMPLE EMBODIMENTS

The terminology used herein is for the purpose of describing particular embodiments only and is not intended to be limiting of the present invention. As used herein, the singular forms “a”, “an” and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

Referencing the drawings, wherein like reference numerals designate identical or corresponding parts throughout the several views, example embodiments of the present patent application are hereafter described.

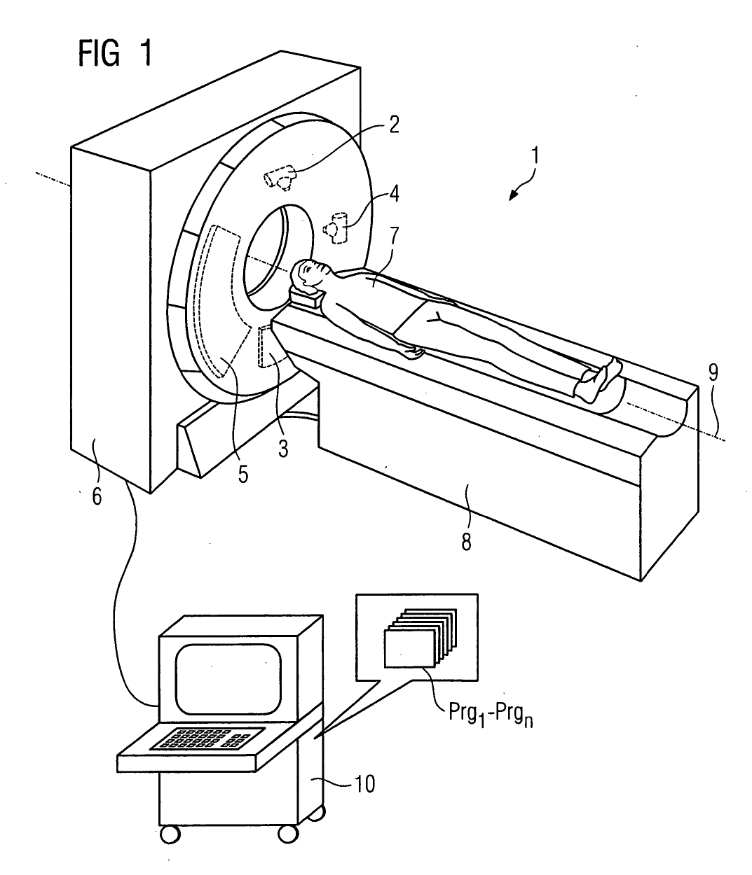

FIG. 1 shows an example computed tomography system 1 having two focus/detector systems having a first focus/detector system FDSA with a first X-ray tube 2 and a detector 3 situated opposite, and a second focus/detector system FDSB to which the second X-ray tube 4 and the detector 5 situated opposite belong. The focus/detector systems 2, 3 and 4, 5 are arranged angularly offset by 90° on a gantry (not illustrated explicitly) in the gantry housing 6, and are moved during scanning of the patient about the system axis 9, while the patient 7 is pushed continuously or sequentially through the scanning region. This purpose is served by a patient couch 8 that can be displaced longitudinally and is driven by the control and computation unit 10.

The control and computation unit 10 is also responsible for controlling and operating the gantry with the two focus/detector systems 2, 3 and 4, 5. Moreover, the absorption data that are obtained by the two focus/detector systems are collected in this control and computation unit 10 and can also be converted thereby by way of the reconstruction method (known per se) into CT image data records or CT volume data records. The programs Prg₁ to Prgₙ illustrated by way of example and in which the method steps according to at least one embodiment of the invention are also depicted are used to this end.

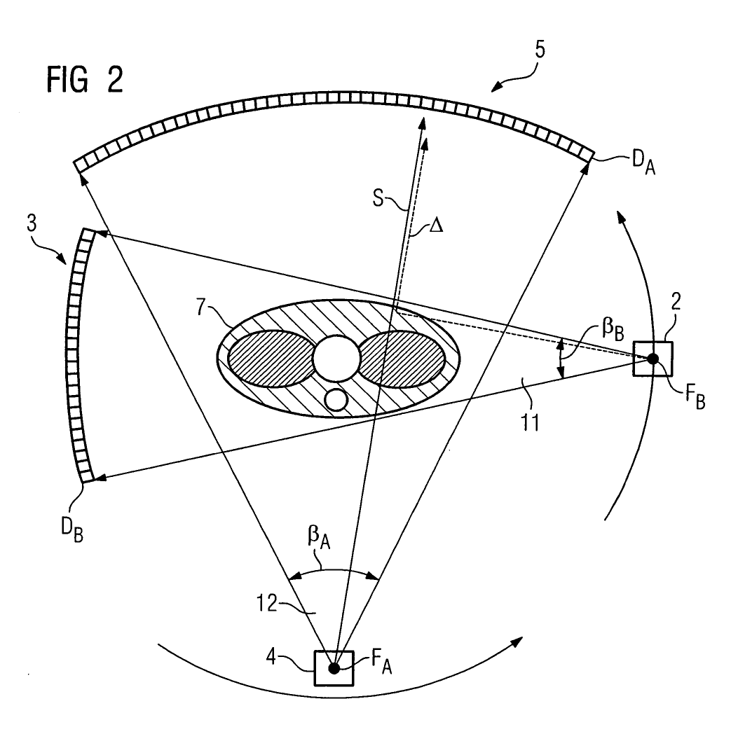

The schematic of FIG. 2 serves for better understanding of the problems of transverse scattering in such a CT system with two focus/detector systems. A patient 7 is illustrated which has a coarsely illustrated inner structure that is scanned by the two focus/detector systems FDSA with the focus F_{A} and the detector D_{A}, and the focus/detector system FDSB, arranged offset therefrom by 80°, with the focus F_{B} and the detector D_{B}. The two assigned X-ray tubes 2 and 4 are indicated for a better orientation with reference to FIG. 1 and the detectors D_{A} and D_{B}, which are illustrated here only as a row of detector elements, are assigned the reference numerals 5 and 3, respectively. The fan angles of the ray fans used are represented by β_{A} and β_{B}, the beam cones 12 and 11 being formed from the foci F_{A} and F_{B}, respectively.

The direction of revolution of the two focus/detector systems is likewise indicated.

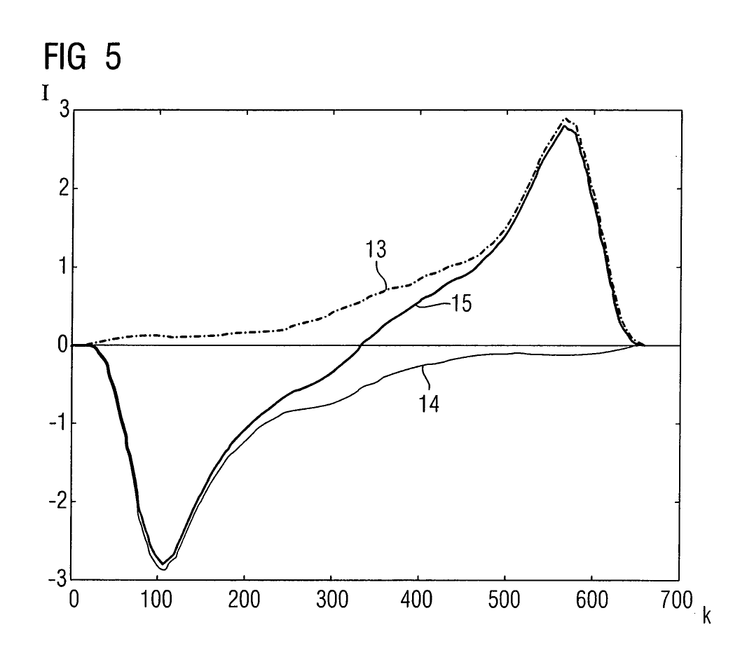

It is seen from a consideration of a direct ray emanating from the focus F_{A} toward a detector element of the detector D_{A} that if both focus/detector systems are in operation, a scattered radiation Δ simultaneously occurs that likewise makes a contribution to the measured intensity at the same detector element at which the intensity I of the ray S is measured. The inventors have recognized here that the principal fraction of the scattered radiation emanates substantially from the surface layer of the scanned object such that scattered radiation parallel to the ray S is not, for example, produced from all depth layers of the patient, but that scattered radiation fractions are chiefly produced on the side of the patient facing the detector D_{A}. The result of these geometric relationships is that when parallel projections are being considered the scattered radiation fraction has an asymmetric profile seen over the number of channels, as is illustrated by way of example in FIG. 5 in the profile of the curve 13 and, in a fashion complementary thereto, in the profile of the curve 14.

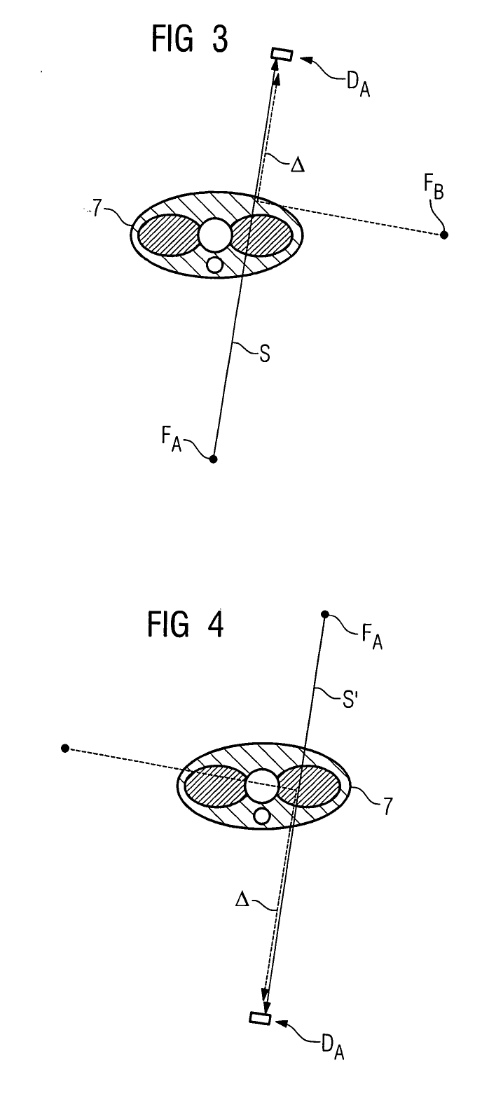

Looking, now, at an individual scanning ray S in FIG. 3 which emanates from a focus F_{A} and runs to a detector element of the detector D_{A}, and considering where the scattered radiation that is generated by the focus F_{B} offset by 90° must in essence be produced, the result is a principal production location of the scattered radiation such as is shown in FIG. 3 by the dashed line of the scattered radiation fraction Δ.

In this context, FIG. 4 shows the ray S′, running in complementary fashion, after the two focus/detector systems have been rotated by 180°. During calculation of the attenuation over this ray profile, the ray S′ would actually have to exhibit the same intensity I as the ray S from FIG. 3. However, since the focus F_{B} in FIG. 4 is arranged on the other side, and the scattered radiation has a substantially lower intensity over the dotted path of the ray from F_{B} to D_{A}, it is possible to determine a substantial fraction of the scattered radiation that is measured in FIG. 3 solely from the difference formation of the two intensities of the ray and the ray S′ arranged in a complementary fashion thereto.

It is possible in this way to form in principle for all the rays a difference between the direct ray S and a ray S′ arranged in a fashion complementary thereto, measured with the aid of the same detector system but in a fashion offset by 180°, in which case whenever the intensity I of the direct ray is greater than the intensity I′ of the complementary ray S′ it can be assumed that this fraction is a scattered radiation fraction such that this fraction can be subtracted from the intensity I of the ray S.

Although it is to be pointed out that this method cannot remove 100% of all scattered radiation fractions from the measured data, nevertheless the largest fraction is eliminated by this computation method.

FIG. 5 shows a profile, calculated by a Monte-Carlo simulation, of the scattered radiation of a direct and an indirect parallel projection, the channels being plotted on the abscissa, and the measured intensity I being plotted on the ordinate in arbitrary units. Here, the profile of the scattered radiation of the direct projection is denoted by the reference 13, and the intensities of the scattered radiation complementary thereto are denoted by the profile 14. The negative intensity shown here is intended merely to represent that what is involved is intensities that are arranged in opposite directions, whereas, of course, only positive intensities occur during the actual measurement of intensity. Subtracting the two intensity profiles 13 and 14 produces the curve 15, all the positive values of the curve 15 being subtracted in accordance with the invention from the entire profile of the intensities of the direct projection, and the scattered radiation correction being carried out thereby. The negative fraction of this curve 15 is ignored in this case.

Thus, overall, at least one embodiment of the invention proposes a method for scattered radiation correction of a CT system having two simultaneously operated focus/detector systems, arranged angularly offset from one another on a rotatable gantry, in which in order to scan an object the two focus/detector systems arranged angularly offset from one another scan the object by virtue of the fact that they rotate about a system axis of the CT system, and a multiplicity of absorption values of individual rays are determined from the measured attenuations of the radiation of the foci and the measured values are subjected to scattered radiation correction, the positive differences for the direct rays S being determined in channelwise fashion from the intensity values I of the direct rays S and the intensity values I′ of the “complementary” rays S′ removed by 180° and this positive difference Δ=I−I′ is subtracted as scattered radiation correction from the intensity value I of the direct ray S in order thereby to determine the attenuation values and to reconstruct CT tomograms or CT volume data from these in a known way.

It is self-evident that the abovenamed features of embodiments of the invention can be used not only in the respectively specified combination, but also in other combinations or on their own, without departing from the framework of the invention.

Thus, overall, at least one embodiment of the invention proposes a method for scattered radiation correction of a CT system in the case of which two focus/detector systems are arranged angularly offset from one another on a rotatable gantry and are operated simultaneously, in which in order to scan an object the two focus/detector systems arranged angularly offset from one another scan the object by virtue of the fact that they rotate about a system axis of the CT system, and a multiplicity of absorption values of individual rays are determined from the measured attenuations of the radiation of the foci and the measured values are subjected to scattered radiation correction, the positive differences for the direct rays being determined in channelwise fashion from the intensity values of the direct rays and the intensity values of the complementary rays removed by 180° and this positive difference is subtracted as scattered radiation correction from the intensity value of the direct ray in order thereby to determine the actual attenuation values and to reconstruct CT tomograms or CT volume data from these in a known way.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for scattered radiation correction of a CT system including two simultaneously operated focus/detector systems, arranged angularly offset from one another on a rotatable gantry, the method comprising:
scanning an object, using the focus/detector systems arranged angularly offset from one another, by rotating the systems about a system axis of the CT system;
determining a multiplicity of absorption values of individual rays from the measured attenuations of the radiation of the foci; and
reconstructing at least one of CT pictures and CT volume data of the object using the determined absorption data, wherein for each direct ray of a focus/detector system, an oppositely directed complementary ray of the same focus/detector system offset by 180° is determined and, if it cannot be determined directly from the detector data, it is determined by interpolation of absorption data of rays of the focus/detector system that are situated and oriented in a spatially similar fashion, wherein the intensity value of the complementary ray is subtracted from the attenuated intensity value of each direct ray, wherein positive fractions, of the difference between the intensity values of the direct ray and the intensity value of the complementary ray, are interpreted as scattered radiation fraction and subtracted from the intensity value of the direct ray, a corrected absorption value of the direct ray being determined therefrom, and wherein at least one of CT pictures and CT volume data is reconstructed from the corrected absorption values.

2. A method for scattered radiation correction of a CT system including two simultaneously operated focus/detector systems, arranged angularly offset from one another on a rotatable gantry, the method comprising:
scanning an object, using the focus/detector systems arranged angularly offset from one another, by rotating the focus/detector systems about a system axis of the CT system;
calculating, from measured attenuations of the radiation of the foci, a multiplicity of parallel projections, from absorption values, from intensity values attenuated by the object and unattenuated, and subjecting the measured values to scattered radiation correction; and
reconstructing CT pictures of the object with the aid of the parallel projections, wherein for each direct parallel projection of a focus/detector system that originates exclusively from absorption data, measured in the same direction, of a focus/detector system, an oppositely directed, complementary parallel projection of the same focus/detector system is at least one of determined and, if it cannot be taken directly from the detector data, interpolated by interpolation of absorption data of rays of the same focus/detector system situated and oriented in a spatially similar fashion, wherein the values of the attenuated intensity values of the complementary parallel projection are subtracted from the attenuated intensity values of each direct parallel projection in channel-wise fashion, wherein the channel-wise existing differences of positive sign are interpreted as the scattered radiation fraction and are subtracted from the direct parallel projection in channel-wise fashion for the purpose of scattered radiation correction, and wherein at least one of CT pictures and CT volume data are reconstructed from the corrected projection data.

3. The method as claimed in claim 1, wherein absorption data of the same focus/detector system are exclusively used for the reconstruction.

4. The method as claimed in claim 1, wherein absorption data of the two focus/detector systems are mixed for the reconstruction.

5. The method as claimed in claim 1, wherein at least one of a calibration, a normalization to a dose monitor value, a radiation hardening correction, a channel correction and a water scaling is carried out before the scattered radiation correction is carried out for each focus/detector system.

6. The method as claimed in claim 1, wherein the focus/detector systems are normalized to one another before the scattered radiation correction is carried out.

7. The method as claimed in claim 1, wherein the scattered radiation fractions are extrapolated in the channel region of the projections in which the signals of the scattered radiation of the direct and complementary rays cancel one another.

8. A CT system, comprising:
at least two simultaneously operated focus/detector systems arranged angularly offset from one another on a rotatable gantry; and
at least one control and computation unit including computer programs to control operation of the CT system and to reconstruct at least one of CT images and CT volume data, at least one computer program including a program code that, when executed,
calculates, from measured attenuations of the radiation of the foci, a multiplicity of parallel projections, from absorption values, from intensity values attenuated by the object and unattenuated, and subjects the measured values to scattered radiation correction, wherein for each direct parallel projection of a focus/detector system that originates exclusively from absorption data, measured in the same direction, of a focus/detector system, an oppositely directed, complementary parallel projection of the same focus/detector system is at least one of determined and, if it cannot be taken directly from the detector data, interpolated by interpolation of absorption data of rays of the same focus/detector system situated and oriented in a spatially similar fashion, wherein the values of the attenuated intensity values of the complementary parallel projection are subtracted from the attenuated intensity values of each direct parallel projection in channel-wise fashion, wherein the channel-wise existing differences of positive sign are interpreted as the scattered radiation fraction and are subtracted from the direct parallel projection in channel-wise fashion for the purpose of scattered radiation correction, and wherein at least one of CT pictures and CT volume data are reconstructed from the corrected projection data.

9. The method as claimed in claim 2, wherein absorption data of the same focus/detector system are exclusively used for the reconstruction.

10. The method as claimed in claim 2, wherein absorption data of the two focus/detector systems are mixed for the reconstruction.

11. The method as claimed in claim 2, wherein at least one of a calibration, a normalization to a dose monitor value, a radiation hardening correction, a channel correction and a water scaling is carried out before the scattered radiation correction is carried out for each focus/detector system.

12. The method as claimed in claim 2, wherein the focus/detector systems are normalized to one another before the scattered radiation correction is carried out.

13. The method as claimed in claim 2, wherein the scattered radiation fractions are extrapolated in the channel region of the projections in which the signals of the scattered radiation of the direct and complementary rays cancel one another.

14. A CT system, comprising:
at least two simultaneously operated focus/detector systems arranged angularly offset from one another on a rotatable gantry; and
at least one control and computation unit including computer programs to control operation of the CT system and to reconstruct at least one of CT images and CT volume data, at least one computer program including a program code that, when executed,
determines a multiplicity of absorption values of individual rays from the measured attenuations of the radiation of the foci, wherein for each direct ray of a focus/detector system, an oppositely directed complementary ray of the same focus/detector system offset by 180° is determined and, if it cannot be determined directly from the detector data, it is determined by interpolation of absorption data of rays of the focus/detector system that are situated and oriented in a spatially similar fashion, wherein the intensity value of the complementary ray is subtracted from the attenuated intensity value of each direct ray, wherein positive fractions, of the difference between the intensity values of the direct ray and the intensity value of the complementary ray, are interpreted as scattered radiation fraction and subtracted from the intensity value of the direct ray, a corrected absorption value of the direct ray being determined therefrom, and wherein at least one of CT pictures and CT volume data is reconstructed from the corrected absorption values.

15. A CT system, comprising:
at least two simultaneously operated focus/detector systems arranged angularly offset from one another on a rotatable gantry; and
means for calculating, from measured attenuations of the radiation of the foci, a multiplicity of parallel projections, from absorption values, from intensity values attenuated by the object and unattenuated, and subjects the measured values to scattered radiation correction, wherein for each direct parallel projection of a focus/detector system that originates exclusively from absorption data, measured in the same direction, of a focus/detector system, an oppositely directed, complementary parallel projection of the same focus/detector system is at least one of determined and, if it cannot be taken directly from the detector data, interpolated by interpolation of absorption data of rays of the same focus/detector system situated and oriented in a spatially similar fashion, wherein the values of the attenuated intensity values of the complementary parallel projection are subtracted from the attenuated intensity values of each direct parallel projection in channel-wise fashion, wherein the channel-wise existing differences of positive sign are interpreted as the scattered radiation fraction and are subtracted from the direct parallel projection in channel-wise fashion for the purpose of scattered radiation correction, and wherein at least one of CT pictures and CT volume data are reconstructed from the corrected projection data.

16. A CT system, comprising:
at least two simultaneously operated focus/detector systems arranged angularly offset from one another on a rotatable gantry; and
means for determining a multiplicity of absorption values of individual rays from the measured attenuations of the radiation of the foci, wherein for each direct ray of a focus/detector system, an oppositely directed complementary ray of the same focus/detector system offset by 180° is determined and, if it cannot be determined directly from the detector data, it is determined by interpolation of absorption data of rays of the focus/detector system that are situated and oriented in a spatially similar fashion, wherein the intensity value of the complementary ray is subtracted from the attenuated intensity value of each direct ray, wherein positive fractions, of the difference between the intensity values of the direct ray and the intensity value of the complementary ray, are interpreted as scattered radiation fraction and subtracted from the intensity value of the direct ray, a corrected absorption value of the direct ray being determined therefrom, and wherein at least one of CT pictures and CT volume data is reconstructed from the corrected absorption values.

17. A computer readable medium including program segments for, when executed on a computer device of a computed tomography system, causing the computed tomography system to implement the method of claim 1.

18. A computer readable medium including program segments for, when executed on a computer device of a computed tomography system, causing the computed tomography system to implement the method of claim 2.
